(19) United States
(12) Patent Application Publication
(10) Pub. No.: US 2024/0374529 A1
(43) Pub. Date: Nov. 14, 2024

(54) PHARMACEUTICAL COMPOSITION COMPRISING POLYSACCHARIDE

(71) Applicants: HUNG, MIEN-CHIE, TAICHUNG CITY (TW); EVER SUPREME BIO TECHNOLOGY CO. LTD, TAICHUNG CITY (TW)

(72) Inventors: CHIH-SHENG CHIANG, TAICHUNG CITY (TW); LONG-BIN JENG, TAICHUNG CITY (TW); WOEI-CHERNG SHYU, HSINCHU CITY (TW); BO-JIE HUANG, TAICHUNG CITY (TW); MIEN-CHIE HUNG, TAICHUNG CITY (TW)

(73) Assignees: MIEN-CHIE HUNG, TAICHUNG CITY (TW); EVER SUPREME BIO TECHNOLOGY CO. LTD, TAICHUNG CITY (TW)

(21) Appl. No.: 18/291,685

(22) PCT Filed: Jul. 27, 2022
(86) PCT No.: PCT/CN2022/108377

§ 371 Date: Jan. 24, 2024

Related U.S. Application Data
(60) Provisional application No. 63/203,637, filed on Jul. 27, 2021.

(51) Int. Cl. A61K 9/51; A61K 31/337; A61K 45/06; A61P 35/00

(52) U.S. Cl. CPC A61K 9/5161; A61K 9/5123; A61K 9/5153; A61K 31/337; A61K 45/06; A61P 35/00

(57) ABSTRACT

A pharmaceutical composition includes a complex, wherein the complex includes a polysaccharidic shell and a hydrophobic core. The polysaccharidic shell has amphiphilicity to stabilize the oil-water interface and form emulsion and nanoprecipitated-based nanoparticles.

### FIELD OF THE INVENTION

The present invention relates to a field of application of polysaccharide. Particularly, the present disclosure relates a pharmaceutical composition containing a polysaccharide.

### BACKGROUND OF THE INVENTION

Fucoidan is a sulphated polysaccharide which possesses multiple biological activities including antibacterial, antiviral, antitumor, anticoagulant, and antioxidant activities. Fucoidan also shows high affinity toward p-selectin, which holds the potential for targeted delivery of therapeutic compounds toward p-selectin-overexpressed sites such as tumors or unstable atherosclerotic plaque. However, the retention time of fucoidan after administration is extremely short, which presents an obstacle to accumulation of the pure compound at the site of interest. Therefore, even if fucoidan possesses the biological activities, since the above-mentioned obstacle in accumulation, its therapeutic capacity is limited.

An engineered drug delivery system (DDS) is a technology for the targeted delivery and/or controlled release of therapeutic agents at desired tissues/organs. Polysaccharide such as fucoidan allows the formation of complexes with other oppositely charged molecules. Polyelectrolyte complexation is the most commonly used technique for obtaining fucoidan-based particles. One of the most common used materials to complex with fucoidan is chitosan. Positive-charged chitosan can interact with fucoidan and form a self-assemble or layer-by-layer DDS. Other methods are coacervation, ionic cross-linking, self-assembly, and spray-drying.

However, although fucoidan-based particles may be synthesized, fucoidan is not a favorable material for stabilizing the water-oil interface of an emulsion or a nanoprecipitated nanostructure. As most of the side chains on fucoidan have been substituted with sulphate groups, the polysaccharide structure is strongly hydrophilic, lacking the amphiphilicity to stabilize water-oil interfaces. Consider that emulsification and nanoprecipitation are the most eco-effective and mature technologies in the pharma industry, it is important to develop technologies to overcome these issues.

### SUMMARY OF THE INVENTION

In one aspect, the present disclosure provides a complex comprising a polysaccharidic shell and a hydrophobic core, wherein the polysaccharidic shell comprises a sulphated polysaccharide and a compensator having affinity to the sulphated polysaccharide, the hydrophobic core comprises a hydrophobic molecule, and the complex has amphiphilicity to reduce the surface tension and stabilize water-oil interface, especially between the polysaccharidic shell and hydrophobic core.

In one aspect, the present disclosure provides a pharmaceutical composition comprising the complex as described in the disclosure.

In one embodiment, the pharmaceutical composition is an emulsion-based nanoparticle. In one further embodiment, the nanoparticle is a nanoprecipitated nanostructure.

In some embodiments of the disclosure, the sulphated polysaccharide is fucoidan.

In some embodiments of the disclosure, the complex has affinity to p-selectin or a modification thereof.

In one embodiment, fucoidan used in the disclosure has a peak molecular ranging from 10 to 200 kDa. Particular embodiments of fucoidan include, but are not limited to, fucoidan derived from Fucus vesiculosus, Okinawa mozuku, Cladosiphon okamuranus Tokida, Ascophyllum nodosum, Fucus evanescens, Fucus ceranoides, Fucus distichus, Fucus serratus, Fucus spiralis, Ascophyllum mackaii, Pelvetia canaliculate, Silvetia babingtonii and Undaria pinnatifida.

In one embodiment, fucoidan used in the disclosure has a purity ranging from about 60% to about 99%; about 65% to about 95%; about 70% to about 90%; about 75% to about 85%; or about 70% to about 80%.

In one embodiment, fucoidan used in the disclosure has a sulphate content ranging from about 15% to about 40%; about 18% to about 38%; about 20% to about 35%; about 22% to about 32%; or about 25% to about 30%.

In some embodiments of the disclosure, the compensator described herein has a positively charged functional group. In some embodiments of the disclosure, the compensator is a positively charged amino acid. Examples of the positively charged amino acid include, but are not limited to, lysine or a polymerized/copolymerized molecule thereof, arginine or a polymerized/copolymerized molecule thereof, histidine or a polymerized/copolymerized molecule thereof, and glutamine or a polymerized/copolymerized molecule thereof. In some embodiments of the disclosure, the compensator having a positively charged functional group further comprises a hydrophobic domain. Examples of the compensator having a positively charged functional group and a hydrophobic domain include but are not limited to zein, chitosan, protamine, or polyethyleneimine.

In some embodiments of the disclosure, the molar ratio of the sulphated polysaccharide to the compensator having a positively-charged functional group ranging from about 1:0.005 to about 1:200; from about 1:0.01 to about 1:180; from about 1:0.02 to about 1:160; from about 1:0.03 to about 1:140; from about 1:0.04 to about 1:120; from about 1:0.05 to about 1:100; from about 1:0.06 to about 1:80; from about 1:0.07 to about 1:60; from about 1:0.08 to about 1:50; from about 1:0.09 to about 1:20; from about 1:0.1 to about 1:10; from about 1:0.2 to about 1:8; from about 1:0.3 to about 1:7; from about 1:0.4 to about 1:6; from about 1:0.5 to about 1:5; from about 1:0.6 to about 1:6; from about 1:0.7 to about 1:5; from about 1:0.8 to about 1:4; from about 1:0.9 to about 1:3; or from about 1:1 to about 1:2.

In some embodiments of the disclosure, for forming an electrically stable complex, the ratio of the negative charges in the sulphated polysaccharide to the positive charge in the compensator ranges from about 1:0.05 to about 1:3; from about 1:0.06 to about 1:2.5; from about 1:0.07 to about 1:2; from about 1:0.08 to about 1:15; from about 1:0.09 to about 1:1; from about 1:0.1 to about 1:0.95; from about 1:0.2 to about 1:0.9; from about 1:0.3 to about 1:85; from about 1:0.4 to about 1:0.8; from about 1:0.5 to about 1:0.7; or from about 1:0.6 to about 1:0.65.

In some embodiments of the disclosure, the compensator bonds to the sulphated polysaccharide through a hydrogen bond. In some embodiments of the disclosure, the compensator comprises an amine-containing ligand, a carboxylic acid group or an oxygen acceptor. Examples of the compensator bonding to the sulphated polysaccharide through a hydrogen bond include, but are not limited to, oxidized dextran, polyethylene glycol (PEG), chemically-modified PEG, polydextrose, polysorbate 20, polysorbate 80, polyvinyl acetate, polyvinyl alcohol (PVA), PLURONIC® F68, PLURONIC® F123, PLURONIC® F127, polyvinyl alcohol, and propylene glycol alginate. Examples of the chemically-modified PEG include, but are not limited to, NH₂-PEG or COOH-PEG.

In some embodiments of the disclosure, the molar ratio of the sulphated polysaccharide to the compensator bonding to the sulphated polysaccharide through a hydrogen bond ranges from about 1:0.01 to about 1:100; from about 1:0.01 to about 1:90; from about 1:0.02 to about 1:80; from about 1:0.03 to about 1:70; from about 1:0.04 to about 1:60; from about 1:0.05 to about 1:50; from about 1:0.06 to about 1:40; from about 1:0.07 to about 1:30; from about 1:0.08 to about 1:20; from about 1:0.09 to about 1:10; from about 1:0. 1 to about 1:9; from about 1:0.2 to about 1:8; from about 1:0.3 to about 1:7; from about 1:0.4 to about 1:6; from about 1:0.5 to about 1:5; from about 1:0.6 to about 1:6; from about 1:0.7 to about 1:5; from about 1:0.8 to about 1:4; from about 1:0.9 to about 1:3; or from about 1:1 to about 1:2.

In some embodiments of the disclosure, the compensator described herein is p-selectin or a modification thereof.

In some embodiments of the disclosure, the molar ratio of the sulphated polysaccharide to the compensator as p-selectin ranges from about 1:0.1 to about 1:100; from about 1:0.5 to about 1:95; from about 1:1 to about 1:90; from about 1:5 to about 1:85; from about 1:10 to about 1:80; from about 1:15 to about 1:75; from about 1:20 to about 1:70; from about 1:25 to about 1:65; from about 1:30 to about 1:60; from about 1:35 to about 1:55; from about 1:40 to about 1:50.

As described herein, the hydrophobic core comprises a. In some embodiments of the disclosure, the hydrophobic core is a lipid, an oil, a hydrophobic polymer, or a polypeptide. In one embodiment of the disclosure, the hydrophobic core is co-encapsulated with the therapeutic agent in the pharmaceutical composition. Examples of the oil include, but are not limited to, vegetable oils, labrafac, soybean oil, castor oil, olive oil, Nigella sativa oil, garlic oil, Echium oil, cottonseed oil, peanut oil, sesame oil, anise oil, cinnamon oil, coconut oil, corn oil, PEG-60 hydrogenated castor oil, and polyoxyl 35 castor oil.

In some embodiments, the hydrophobic core comprises one or more types of lipids. Examples of the lipids include but are not limited to nonionic/ionic lipids such as tristearin, phosphate lipid, egg phospholipid, stearic acid, lecithin, cholesterol, hydrogenated soy phosphatidylcholine, 1,2-distearoyl-sn-glycero-3-phosphocholine (DSPC), 1,2-Distearoyl-sn-glycero-3-phosphorylethanolamine (DSPE), DSPE-PEG, 1,2-dioleoyl-3-trimethylammoniumpropane (DOTAP), dimethyldioctadecylammonium (DDA), and 1,2-dimyristoylrac-glycero-3 (DMG)-PEG.

In some embodiments, the hydrophobic core comprises one or more types of hydrophobic polymers. Examples of the hydrophobic polymer include, but are not limited to, poly lactic acid (PLA), poly glycolic acid (PGA), poly lactic-co-glycolic acid (PLGA).

In some embodiments, the hydrophobic core described herein includes more than one kind of materials. In some embodiments of the disclosure, the hydrophobic core comprises lipids and hydrophobic polymers.

In some embodiments, the polysaccharidic shell described herein includes more than one kind of compensator. Examples of the polysaccharidic shell include but are not limited to a complex comprising fucoidan, PVA, and lysine. For example, based on 10 mg of fucoidan, the weight of PVA ranges from about 0.01 mg to about 2 mg; 0.05 mg to about 1.95 mg: 0.10 mg to about 1.90 mg; 0.15 mg to about 1.85 mg; 0.2 mg to about 1.8 mg; 0.25 mg to about 1.75 mg; 0.30 mg to about 1.70 mg; 0.35 mg to about 1.75 mg;0.40 mg to about 1.70 mg; 0.45 mg to about 1.65 mg; 0.45 mg to about 1.65 mg; 0.50 mg to about 1.60 mg; 0.55 mg to about 1.55 mg; 0.60 mg to about 1.50 mg; 0.65 mg to about 1.55 mg; 0.70 mg to about 1.50 mg; 0.75 mg to about 1.45 mg: 0.80 mg to about 1.4 mg; 0.85 mg to about 1.35 mg; 0.9 mg to about 1.3 mg; 0.95 mg to about 1.25 mg; 1.0 mg to about 1.20 mg; or 1.05 mg to about 1.15 mg. For example, based on 10 mg of fucoidan, the weight of lysine ranges from about 0.3 mg to about 5 mg; about 0.35 mg to about 4.95 mg; about 0.40 mg to about 4.90 mg; about 0.45 mg to about 4.85 mg; about 0.5 mg to about 4.8 mg; about 0.55 mg to about 4.75 mg; about 0.6 mg to about 4.7 mg; about 0.65 mg to about 4.65 mg; about 0.70 mg to about 4.60 mg; about 0.70 mg to about 4.55 mg; about 0.75 mg to about 4.5 mg; about 0.80 mg to about 4.45 mg; about 0.85 mg to about 4.40 mg; about 0.90 mg to about 4.45 mg; about 0.95 mg to about 4.50 mg; about 1.0 mg to about 4.45 mg; about 1.5 mg to about 4.4 mg; about 2.0 mg to about 4.3 mg; about 2.5 mg to about 4 mg; or about 3.0 mg to about 4 mg.

In one embodiment of the disclosure, the complex comprises fucoidan, PLGA, and lysine. In some embodiments of the disclosure, the molar ratio between fucoidan to PLGA is from about 1:3 to about 1:25; about 1:4 to about 1:24; about 1:5 to about 1:23; about 1:6 to about 1:22; about 1:7 to about 1:21; about 1:8 to about 1:20; about 1:9 to about 1:19; about 1:10 to about 1:18; about 1:11 to about 1:17; about 1:12 to about 1:16; about 1:13 to about 1:17; about 1:14 to about 1:16; or about 1:15. In some embodiments of the disclosure, the molar ratio between fucoidan to lysine is from about 1:40 to about 1:160; about 1:50 to about 1:150; about 1:60 to about 1:140; about 1:70 to about 1:130; about 1:80 to about 1:120; about 1:90 to about 1:110; or about 1:100 to about 1:105. In some embodiments of the disclosure, the complex comprising fucoidan, PLGA, and lysine further comprises soybean oil. In some embodiments of the disclosure, the molar ratio between fucoidan to soybean oil is from about 1:6 to about 1:26; about 1:7 to about 1:25; about 1:8 to about 1:24; about 1:9 to about 1:23; about 1:10 to about 1:22; about 1:11 to about 1:21; about 1:12 to about 1:20; about 1:13 to about 1:19; about 1:14 to about 1:18; about 1:15 to about 1:17; or about 1:16.

In some embodiments, the pharmaceutical composition further comprises a therapeutic agent. In one embodiment of the disclosure, the therapeutic agent is encapsulated in the complex. Examples of the therapeutic agent include, but are not limited to, an anticancer drug, an anti-inflammation drug, a drug for stroke medication, an immune modulator, a nucleic acid molecule, an antibacterial drug, an antiviral drug, an anticoagulant drug, or an antioxidant drug.

Examples of the anticancer drug include, but are not limited to, bleomycin, cisplatin, carboplatin, cytarabine, docetaxel, doxorubicin, daunorubicin, epirubicin, fluorouracil, gemcitabine, irinotecan, leuprorelin, oxaliplatin, paclitaxel, pemetrexed, topotecan, vinorelbine, or vinblastine.

Examples of the anti-inflammation drug include, but are not limited to, ibuprofen, naproxen sodium, diclofenac potassium, celecoxib, sulindac, oxaprozin, piroxicam, or indomethacin.

Examples of the drug for stroke medication, include but are not limited to, tissue plasminogen activator (tPA), warfarin, clopidogrel, aspirin, atorvastatin, fluvastatin, lovastatin, pitavastatin, pravastatin, rosuvastatin, or simvastatin.

Examples of the immune modulator include, but are not limited to, cytokines, thalidomide, lenalidomide, pomalidomide, or imiquimod.

Examples of the nucleic acid include, but are not limited to, plasmid DNA, messenger RNA (mRNA), RNA inhibitor (RNAi), small interfering RNA (siRNA), aptamer, or microRNA. In some embodiments of the disclosure, the nucleic acid is plasmid DNA, siRNA, or aptamer.

In some embodiments of the disclosure, the loading capacity of the complex for the therapeutic agent ranges from about 1% to about 30%; from about 2% to about 28%; from about 3% to about 26%; from about 4% to about 24%; from about 6% to about 22%; from about 8% to about 20%; from about 10% to about 18%; from about 12% to about 16%; from about 12% to about 15%; or from about 12% to about 14%.

In some embodiments of the disclosure, the pharmaceutical composition shows the ability to target CD62P (p-selectin) in tumor microenvironment to improve the therapeutic efficacy of the encapsulated therapeutic agent in CD62P positive cancer types such as breast cancer, lymphoma, lung cancer, bladder cancer, ovarian cancer, and pancreatic cancer.

The present disclosure also provides a method for treating a disease in a subject in need of such treatment comprising administrating to the subject the pharmaceutical composition as described herein.

The present disclosure also provides use of the pharmaceutical composition as described herein in the manufacture of a medicament for treating a disease in a subject in need of such treatment.

In some embodiments of the disclosure, the therapeutic agent is an anticancer drug and the disease is selected from the group consisting of breast cancer, lymphoma, lung cancer, bladder cancer, ovarian cancer, and pancreatic cancer.

### BRIEF DESCRIPTION OF THE DRAWINGS

FIG. 1 shows the particle size of the pharmaceutical composition comprising fucoidan, lysine and PLGA in Example 1 and docetaxel.

FIG. 2 shows the colloidal stability of the pharmaceutical composition in Example 3 comprising fucoidan, lysine, PLGA and docetaxel.

FIG. 3A shows that the fucoidan-based DDSs described in Example 1 extend the median survival of 4T1-3 bearing mice. FIG. 3B shows that the fucoidan-based DDSs described in Example 1 extend the median survival of SKOV-3 bearing mice.

FIG. 4 shows the particle size of the pharmaceutical composition in Example 4 comprising fucoidan, lysine, soybean oil, PLGA and docetaxel.

FIG. 5 shows the stability of the pharmaceutical composition in Example 4 comprising fucoidan, lysine, soybean oil, PLGA and docetaxel.

### DETAILED DESCRIPTION OF THE INVENTION

Unless defined otherwise, all scientific or technical terms used herein have the same meaning as understood by those of ordinary skill in the art to which the present invention belongs. Any method and material similar or equivalent to those described herein can be understood and used by those of ordinary skill in the art to practice the present invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and claims of the present invention are approximate and can vary depending upon the desired properties sought to be obtained by the present invention.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

“About” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

As used in the present invention, the term “pharmaceutical composition” means a mixture containing a therapeutic agent administered to a mammal, for example a human, for preventing, treating, or eliminating a particular disease or pathological condition that the mammal suffers.

As used herein, the term “complex” refers to a material comprising two or more materials having different physical or chemical properties, wherein the complex has properties different from individual materials constituting the complex, and wherein the individual materials are macroscopically or microscopically separated and distinguishable from each other in a finished structure of the complex.

As used herein, the term “amphiphilicity” refers to the property of one substance having both a hydrophobic site and a hydrophilic site. For example, when the medium is water, a substance having amphiphilicity forms micelle particles and the particles can be observed. In some embodiments of the disclosure, a molecule has amphiphilicity is able to reduce the surface tension and stabilize water-oil interface.

As used herein, the term “affinity” refers to the strength of the binding interaction between two molecules. Generally, binding affinity refers to the strength of the sum total of non-covalent interactions between a molecule and its binding partner.

As used herein, the term “loading capacity” refers to a ratio of the loaded therapeutic agent in a pharmaceutical composition to the whole pharmaceutical composition.

The term “treating” or “treatment” as used herein denotes reversing, alleviating, inhibiting the progress of, or improving the disorder, disease or condition to which such term applies, or one or more symptoms of such disorder, disease or condition.

As used in the present invention, the term “therapeutic agent” means any compound, substance, drug, drug or active ingredient having a therapeutic or pharmacological effect that is suitable for administration to a mammal, for example a human.

As used herein, the term “subject” refers to any mammal potentially being treated with the disclosed compositions. The subject can be a vertebrate, for example, a mammal. In some embodiments, the subject is, for example, a human, a primate, a dog, a cat, a horse, a cow, a pig, a rodent, such as for example a rat or mouse. Typically, the subject can be a human. The subject can be symptomatic or asymptomatic. The term does not denote a particular age or sex. Thus, adult and newborn subjects, whether male or female, are intended to be covered. A subject can include a control subject or a test subject.

As used herein, the term “in need of treatment” refers to a judgment made by a caregiver (e.g., physician, nurse, nurse practitioner, or individual in the case of humans; veterinarian in the case of animals, including non-human mammals) that a subject requires or from whom the subject will benefit from treatment. This judgment is made based on a variety of factors that are in the realm of a caregiver's expertise, but that includes the knowledge that the subject is ill, or will be ill, as the result of a condition that is treatable by the compounds of the present disclosure.

As used herein, “polysaccharide” may refer to a naturally occurring full length polysaccharide molecule, a mixture of any combinations of hydrolysis products (including monosaccharide, oligosaccharide and polysaccharide species) of a full-length polysaccharide molecule, any chemically modified or functionalized derivative of the full-length polysaccharide molecule or its hydrolysis product, or any combinations thereof. The polysaccharide may be linear or branched, a single chemical species or a mixture of related chemical species (such as molecules with the same basic monosaccharide units, but different number of repeats). As used herein, “sulphated polysaccharide” refers to a polysaccharide where at least one monosaccharide is substituted with a sulphate group. In one embodiment, the sulphated polysaccharide is a polysaccharide where at least one sugar ring is substituted with a sulphate group.

Fucoidan, a sulphated polysaccharide, has multiple biological effects. Fucoidan also possesses high biocompatibility. Thus, it has been used as building blocks of a DDS for improving drug delivery. However, fucoidan lacks amphiphilicity, making it an unfavorable surfactant to stabilize oil-in-water (O/W) or water-in-oil (W/O) interfaces. The structure of fucoidan mainly contains α-fucose residues, while the negative charge of this biopolymer results from the presence of sulfate groups, which are mainly substituted on C-2 and C-4 and occasionally on C-3 positions. Chemical modification of fucoidan's molecular structure has demonstrated improvement in amphiphilicity. However, once the molecular structure is changed, the biological functions such as p-selectin targeting and immunomodulatory effect would be compromised. Therefore, a new strategy to use fucoidan to stabilize an oil-water interface without impairing its biological functions is urgently needed. In some embodiments of the disclosure, the fucoidan is without chemical modification.

In some embodiments of the disclosure, fucoidan is produced by Fucus vesiculosus, Okinawa mozuku, Cladosiphon okamuranus Tokida, Ascophyllum nodosum, Fucus evanescens, Fucus ceranoides, Fucus distichus, Fucus serratus, Fucus spiralis, Ascophyllum mackaii, Pelvetia canaliculate, Silvetia babingtonii and Undaria pinnatifida, and can be purified or partially purified from culture of the organisms. In some embodiments of the disclosure, fucoidan has a peak molecular ranged from 10 kDa to 200 kDa; from 20 kDa to 180 kDa; from 30 kDa to 160 kDa; from 40 kDa to 140 kDa; from 50 kDa to 120 kDa; from 60 kDa to 100 kDa; from 80 kDa to 90 kDa.

Purity of fucoidan may vary. In one embodiment, fucoidan has a purity ranging from about 60% to about 99%; about 65% to about 95%; about 70% to about 90%; about 75% to about 85%; or about 70% to about 80%.

Not willing limited by theory, it is believed that the sulfate content of the sulphated polysaccharide may play a role in the complex according to the disclosure. In one embodiment, fucoidan has a sulphate content ranging from about 15% to about 40%; about 18% to about 38%; about 20% to about 35%; about 22% to about 32%; or about 25% to about 30%.

Accordingly, the present disclosure provides a pharmaceutical composition comprising a complex comprising a polysaccharidic shell and a hydrophobic core, wherein the polysaccharidic shell comprises a sulphated polysaccharide and a compensator having affinity to the sulphated polysaccharide, the hydrophobic core comprises one or multiple hydrophobic molecules.

The present disclosure also provides a method for treating a disease in a subject in need of such treatment comprising administrating the subject the pharmaceutical composition as described herein.

The compensator employed herein refers to a substance that has high affinity toward the sulphated polysaccharide, such as fucoidan, and is able to form the complex as described in the disclosure. The formation of the complex between the sulphated polysaccharide and the compensator can modulate the physiological properties of the sulphated polysaccharide, thus further conferring the complex with the ability to stabilize oil-water interfaces. Moreover, the complexation between the sulphated polysaccharide and the compensator would not alter the molecular structure of the sulphated polysaccharide, and thus theoretically would not impair the biological functions of the sulphated polysaccharide. Hence, the strategy shows potential in forming a stable drug delivery system with homogeneous size distribution, while retaining or even enhancing the biological functions of fucoidan including antibacterial, antiviral, antitumor, anticoagulant, antioxidant activities, and p-selectin targeting ability. Thus, by facilitating the inherent therapeutic properties, a sulphated polysaccharide-based drug delivery system has the ability to deliver drugs to a diseased site, and further enhance the therapeutic effects.

By utilizing the compensator, the complex is able to stabilize an oil-water interface. Therefore, the pharmaceutical composition can be an emulsion-based or a nanoprecipitated nanoparticles.

The compensator as disclosed herein is physically, chemically, or biologically complementary to the sulphated polysaccharide, and the compensator is capable of complexing with the sulphated polysaccharide for stabilizing interfaces without impairing the biological effects. By complexing with compensators, a sulphated polysaccharide-based DDS can be formed through a simple emulsion process, yielding improved stability and broader applications.

The compensator may be in the form of a small molecule, a protein, a polymer, or a combination thereof, which shows high affinity toward the sulphated polysaccharide due to physical, chemical, or biological interaction forces. In one aspect, the compensator would also possess a hydrophobic domain. Thus, when the compensators are mixed with the sulphated polysaccharide at a certain range of ratio, and a certain defined pH value, the complexation would provide the capacity of amphiphilicity to stabilize interfaces. Accordingly, the compensator can shore up the weakness of using hydrophilic the sulphated polysaccharide alone. Note that an emulsion or nanoprecipitation is processed, there is generally a shear stress to mix solutions in different phases into a homogeneous solution. It is required that the compensator and its interaction force with the sulphated polysaccharide be higher than the shear stress, so that the sulphated polysaccharide-compensator complexation can facilitate the stabilization of interfaces without tearing them apart from each other during emulsion.

Examples of the compensator include but are not limited to a physical compensator, a chemical compensator, or a biological compensator.

Examples of physical forces applied in the physical compensator include but are not limited to an electrostatic interaction or a hydrophobic interaction.

The sulphated polysaccharide contains sulphate, making it a strongly negative-charged molecule. A basic compensator that contains positive charges and optionally hydrophobic domains are favorable options to interact with the sulphated polysaccharide and form the complex. Positively-charged amino acids including lysine, arginine, histidine, glutamine and their polymerized/copolymerized molecules can complex with the sulphated polysaccharide by electrostatic force and stabilize O/W and W/O interfaces to provide a smaller particle size after emulsion. Zein, chitosan, protamine, polyethyleneimine (PEI), amine polyethylene glycol (PEG), amine-terminated poly(ethylene oxide) (PEO) and poly(epsilon-caprolactone) (PCL) and other materials/molecules that contain positive-charged functional groups with hydrophobic domains might also serve as compensators for the sulphated polysaccharide, and form complexation to stabilize particle interfaces.

In some embodiments of the disclosure, the range of molar ratio between fucoidan and lysine that can stabilize the O/W and W/O interface and become a stable formulation is from about 1:10 to about 1:160; between fucoidan and arginine is from about 1:0.005 to about 1:5; between fucoidan and histidine is from about 1:0.005 to about 1:5; between fucoidan and glutamine is from about 1:0.005 to about 1:5; between fucoidan and zein is from about 1:0.002 to about 1:10; between fucoidan and chitosan is from about 1:0.05 to about 1:50; between fucoidan and protamine is from about 1:0.02 to about 1:100; between fucoidan and polyethyleneimine is from about 1:0.01 to about 1:100.

In some embodiments of the disclosure, the ratio for negative charges in the sulphated polysaccharide and positive charges in the compensator is from about 1:0.05 to about 1:3.

In one embodiment of the disclosure, hydrogen bonding is applied to the chemical compensator. There are abundant hydroxyl groups on the sulphated polysaccharide, which possesses both hydrogen-bonding donor and acceptor sites that form two types of hydrogen bonds concurrently. Therefore, hydrogen bonding can easily form between the sulphated polysaccharide and a wide variety of materials/molecules that contain hydrogen-bonding donor and acceptor sites. For example, the sulphated polysaccharide can form O—H . . . :N with amine-containing ligand/molecules (i.e., hydrogen donor). The sulphated polysaccharide can also form O—H . . . :O with an acceptor atom like carboxylic acid and other molecules containing oxygen acceptor. It is noted that hydrogen bond is relatively weak; therefore, only when the sulphated polysaccharide is complexed with a hydrogen-bonding donor or acceptor or their combination at a certain ratio, which generates a sufficient force between molecules, can the chemical compensators stick to the sulphated polysaccharide and contribute to the stabilization of water-oil-interface. Molecules such as oxidized dextran, polyethylene glycol (PEG), chemically modified PEG, polydextrose, polysorbate 20, polysorbate 80, polyvinyl acetate, polyvinyl alcohol, Pluronic® F68, Pluronic® F123, Pluronic® F127, polyvinyl alcohol, and propylene glycol alginate that have a hydrogen donor(s) or acceptor(s) have potential to achieve the complexation with fucoidan and stabilize O/W and W/O interface.

As for the biological compensator, it is known that fucoidan is a ligand of p-selectin or a modification thereof, a type-1 transmembrane protein that is encoded by the SELP gene in humans. P-selectin as a protein has hydrophobic domain in its structure. There is a high affinity between fucoidan and p-selectin, and their complexation can therefore become an amphiphilic material for stabilizing oil-water interface. Similarly, fucoidan would have some affinity toward selectins, making the biological interaction between them form complexations, which are predicted to be able to stabilize the oil-water interface. P-selectin can be further chemically modified to increase the portion of hydrophobic side chain or to present functional groups. The modification on P-selectin would result in stronger interaction with fucoidan and provide bridging points for immobilization of targeting ligand or molecules. The complexation of fucoidan and P-selectin, or fucoidan and chemically modified P-selectin, is potentially to stabilize O/W and W/O interfaces.

The materials/molecules mentioned above can be used to complex with the sulphated polysaccharide, providing additive or even synergistic effects in stabilizing the interface to form nano/microparticles with tunable size and structure.

For example, PVA and lysine as different types of compensators were used to complex with fucoidan simultaneously. After emulsion, the formed nanoparticles showed a smaller size (i.e., more compact), more homogeneous size distribution, and higher aqueously colloidal stability. Thus, different types of compensators can be combined with the sulphated polysaccharide at a certain ratio, to form a composition, for optimizing the ability to stabilize O/W and W/O interface and form a required particle size. The weight ratio of the sulphated polysaccharide and different types of compensators that can achieve stabilization of O/W and W/O interfaces vary with the types and number of compensators and their relative composition. For example, the complexation of the sulphated polysaccharide with PVA and lysine can stabilize the O/W and W/O interfaces. For example, based on 10 mg of fucoidan, the weight of PVA ranges from about 0.01 mg to about 2 mg. For example, based on 10 mg of fucoidan, the weight of lysine ranges from about 0.3 mg to about 5 mg.

In one embodiment of the disclosure, the complex comprises fucoidan, PLGA, and lysine. In some embodiments of the disclosure, the molar ratio between fucoidan to PLGA is from about 1:3 to about 1:25; particularly from about 1:3.04 to about 1:22.8. In some embodiments of the disclosure, the molar ratio between fucoidan to lysine is from about 1:40 to about 1:160; particularly from about 1:38.94 to about 1:160. In some embodiments of the disclosure, the complex comprising fucoidan, PLGA, and lysine further comprises soybean oil. In some embodiments of the disclosure, the molar ratio between fucoidan to soybean oil is from about 1:6 to about 1:26; particularly from about 1:6.49 to about 1:26.

As described herein, the hydrophobic core comprises a therapeutic agent. In some embodiments of the disclosure, the hydrophobic core is a lipid or a hydrophobic polymer. Not willing limited by theory, it is believed that with the addition of lipids in the composition, the emulsion presents enhanced surface properties, improved drug delivery and enhanced cell uptake to desired cell/tissue.

In some embodiments of the disclosure, the hydrophobic core comprises an oil. In one embodiment of the disclosure, the hydrophobic core is co-encapsulated with the therapeutic agent in the pharmaceutical composition. Not willing limited by theory, it is believed that with the addition of oil in the composition, the therapeutic agent can be loaded with higher efficiency. Examples of the oil include, but are not limited to, vegetable oils, labrafac, soybean oil, castor oil, olive oil, Nigella sativa oil, garlic oil, Echium oil, cottonseed oil, peanut oil, sesame oil, anise oil, cinnamon oil, coconut oil, corn oil, PEG-60 hydrogenated castor oil, and polyoxyl 35 castor oil.

In some embodiments, the hydrophobic core comprises one or more types of lipids. Examples of the lipids include but are not limited to nonionic/ionic lipids such as tristearin, phosphate lipid, egg phospholipid, stearic acid, lecithin, cholesterol, hydrogenated soy phosphatidylcholine, 1,2-distearoyl-sn-glycero-3-phosphocholine (DSPC), 1,2-Distearoyl-sn-glycero-3-phosphorylethanolamine (DSPE), DSE-PEG, 1,2-dioleoyl-3-trimethylammoniumpropane (DOTAP), dimethyldioctadecylammonium (DDA), and 1,2-dimyristoylrac-glycero-3 (DMG)-PEG.

In some embodiments, the hydrophobic core comprises one or more types of hydrophobic polymers or polypeptides. Examples of the hydrophobic polymer include, but are not limited to, poly lactic acid (PLA), poly glycolic acid (PGA), poly lactic-co-glycolic acid (PLGA) and poly-L-leucine.

In some embodiments, the hydrophobic core described herein includes more than one kind of materials. In some embodiments of the disclosure, the hydrophobic core comprises lipids and hydrophobic polymers.

The sulphated polysaccharide-based DDS is able to carry drugs, alter their pharmacokinetic behavior, improve drug biodistribution, and further improve therapeutic efficacy. Therefore, the pharmaceutical composition as described herein further comprises one or more therapeutic agent. In one embodiment of the disclosure, the therapeutic agent is encapsulated in the complex. Examples of the therapeutic agent that can be incorporated in the fucoidan-compensator formulation include anticancer drug (e.g., bleomycin, cisplatin, carboplatin, cytarabine, docetaxel, doxorubicin, daunorubicin, epirubicin, fluorouracil, gemcitabine, irinotecan, leuprorelin, oxaliplatin, paclitaxel, pemetrexed, topotecan, vinorelbine, vinblastine), anti-inflammation drug (e.g., ibuprofen, naproxen sodium, diclofenac potassium, celecoxib, sulindac, oxaprozin, piroxicam, indomethacin), drug for stroke medication (e.g., tissue plasminogen activator (tPA), warfarin, clopidogrel, aspirin, atorvastatin, fluvastatin, lovastatin, pitavastatin, pravastatin, rosuvastatin, simvastatin), immune modulators (e.g., cytokines, thalidomide, lenalidomide, pomalidomide, and Imiquimod), messenger RNA (mRNA), RNA inhibitor (RNAi), or microRNA.

The following examples are provided to aid those skilled in the art in practicing the present disclosure.

### EXAMPLES

### Example 1 Complex Containing Polysaccharidic Shell Comprising Electrostatic Compensators

The particle size for the emulsion with polysaccharidic shells comprising electrostatic compensators and a hydrophobic PLGA core was analyzed using dynamic light scattering (DLS) and shown in Table 1.

TABLE 1
Improving emulsion properties using electrostatic
compensators of fucoidan.

| Materials | Average Diameter (nm) |
|---|---|
| Fucoidan alone | Cannot form emulsion; diameter cannot be |
| | detected |
| Fucoidan + lysine | 166.4 |
| Fucoidan + polylysine | 168 |
| Fucoidan + arginine | 160 |
| Fucoidan + polyarginine | 171 |
| Fucoidan + histidine | 170 |
| Fucoidan + polyhistidine | 182 |

Positively-charged amino acids including lysine, arginine, histidine, glutamine and their polymerized molecules are shown to complex with the sulphated polysaccharide by electrostatic force and stabilize O/W and W/O interfaces to provide a smaller particle size after emulsion.

Furthermore, the ratio for fucoidan to lysine molecules that are applicable to form emulsion or nanoprecipitation are listed in Table 2.

TABLE 2
Range of molar ratio between fucoidan and lysine
that can stabilize the O/W interface.

| | | |
|---|---|---|
| [SO₃²⁻] in fucoidan:NH₂ | Hydrodynamic | Polydispersity |
| in lysine | Diameter (nm) | index (PDI) |

| | |
|---|---|
| 1:0.02 | Cannot form stable emulsion or |
| | nanoprecipitation |

| | | |
|---|---|---|
| 1:0.16 | 162.2 | 0.195 |
| 1:0.3 | 173.9 | 0.152 |
| 1:0.62 | 178.2 | 0.107 |
| 1:1.2 | 179.9 | 0.152 |
| 1:2.4 | 169.9 | 0.074 |

| | |
|---|---|
| 1:3.6 | Cannot form stable emulsion or |
| | nanoprecipitation |

### Example 2 Complex Containing Polysaccharidic Shell Comprising Different Types of Compensators

The particle size for the emulsion with polysaccharidic shells comprising different types of compensators and a hydrophobic PLGA core was analyzed using dynamic light scattering (DLS) and shown in Table 3.

TABLE 3
Different types of compensators can be combined to achieve additive
effect in stabilizing and forming fucoidan-based nanoparticles.

| | | |
|---|---|---|
| | Average | Polydispersity |
| Materials | Diameter (nm) | index (PDI) |

| | |
|---|---|
| Fucoidan alone | Cannot form stable emulsion or |
| | nanoprecipitation |

| | | |
|---|---|---|
| Fucoidan + Lysine | 163 | 0.264 |
| Fucoidan + PVA | 155 | 0.224 |
| Fucoidan + PVA + lysine | 146 | 0.181 |

### Example 3 Pharmaceutical Composition Comprising the Complex and Therapeutic Agents

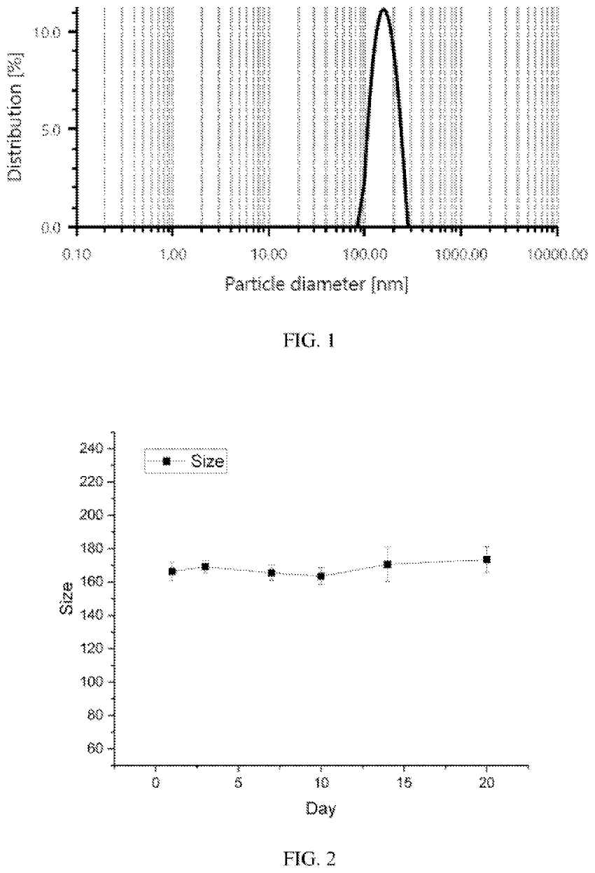

The complex comprises a polysaccharidic shell comprising fucoidan and lysine stabilized the O/W interface to the hydrophobic core comprising PLGA is able to encapsulate docetaxel in the fucoidan-based DDS. The loading capacity of docetaxel can achieve about 15% to about 50%. The particle size for the emulsion was analyzed using DLS and shown in FIG. 1.

The fucoidan-based DDSs described in Example 1 was able to stay colloidal without precipitation at room temperature for at least two weeks. The stability was monitored using DLS and the results are shown in FIG.2.

The fucoidan-based DDSs described in Example 1 showed a more potent cytotoxicity compared with non-formulated DTX in triple negative breast cancer cell line (MDA-MB-231 and 4T1) and pancreatic cancer cell lines (CFPAC-1). The IC50 results are shown in Table 4.

TABLE 4
The IC50 result of the fucoidan-based DDSs
described in Example 1 compared with DTX

| IC50 (nM) | DTX | Fucoidan-based DDSs |
|---|---|---|
| MDA-MB-231 | 18.73 | 2.678 |
| 4T1 | 29.1 | 9.035 |
| CFPAC-1 | 38.84 | 11.15 |

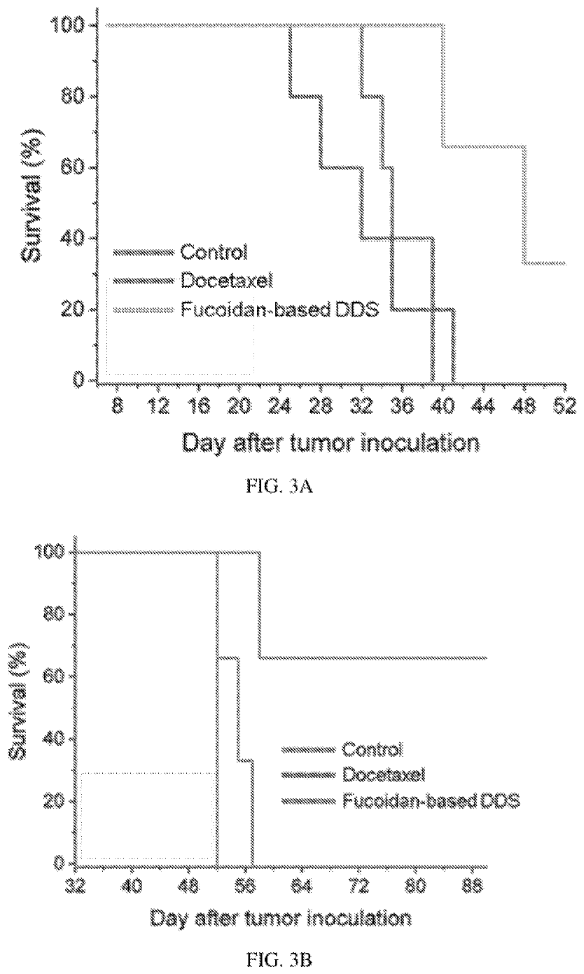

The fucoidan-based DDSs described in Example 1 showed an improved the therapeutic efficacy in a syngeneic 4T1-bearing triple negative breast cancer animal model and SKOV3 ovarian cancer animal model, extending the median survival when compared with docetaxel as shown in FIGS. 3A and 3B.

### Example 4 Pharmaceutical Composition Comprising the Complex and Therapeutic Agents

The complex comprises a polysaccharidic shell comprising fucoidan and lysine stabilized the O/W interface to the hydrophobic core comprising PLGA and soybean oil is able to encapsulate docetaxel in the fucoidan-based DDS. The loading capacity of docetaxel can achieve about 15% to about 45%. The particle size for the emulsion was analyzed using DLS and shown in FIG. 4.

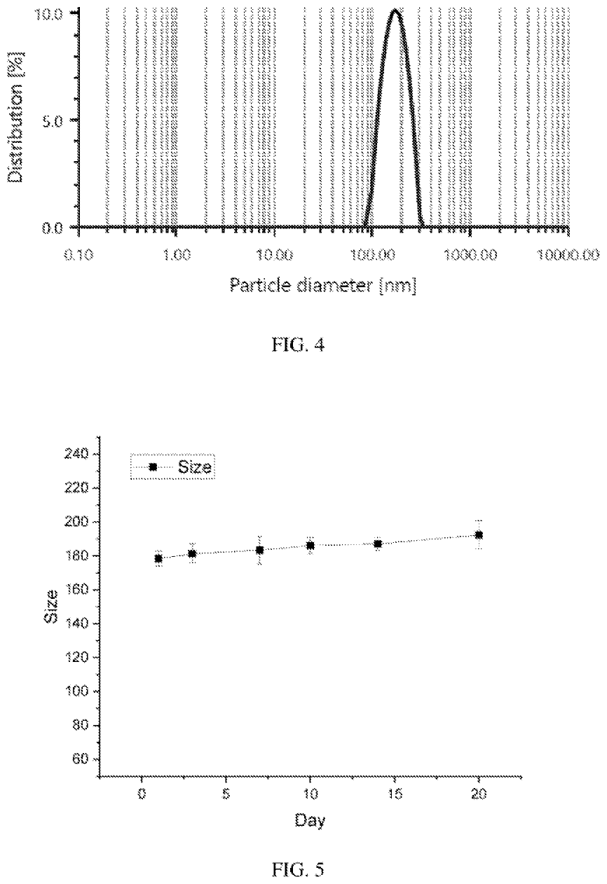

The fucoidan-based DDSs was able to stay colloidal without precipitation at room temperature for at least two weeks. The stability was monitored using DLS and the results are shown in FIG. 5.

The fucoidan-based DDSs showed a more potent cytotoxicity compared with non-formulated DTX in triple negative breast cancer cell line (MDA-MB-231 and 4T1), pancreatic cancer cell lines (CFPAC-1 and BxPC3), and ovarian cancer cell lines (SKOV3). The IC50 results are shown in Table 5.

TABLE 5
The IC50 result of the fucoidan-based DDSs compared with DTX.

| IC50 (nM) | DTX | Fucoidan-based DDSs |
|---|---|---|
| MDA-MB-231 | 18.73 | 9.25 |
| 4T1 | 29.1 | 7.475 |
| BXPC3 | 22.9 | 15.3 |
| CFPAC-1 | 38.84 | 4.9 |
| SKOV3 | 10.09 | 9.778 |

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

1. A complex comprising a polysaccharidic shell and a hydrophobic core, wherein the polysaccharidic shell comprises a sulphated polysaccharide and a compensator having affinity to the sulphated polysaccharide, and the hydrophobic core comprises a hydrophobic molecule.

2. The complex according to claim 1, wherein the sulphated polysaccharide is fucoidan.

3. The complex according to claim 2, wherein the fucoidan has a peak molecular ranging from 10 to 200 kDa; a purity ranging from about 60% to about 99%; and/or a sulphate content ranging from about 15% to about 40%.

4. The complex according to claim 1, wherein the compensator is selected from the group consisting of a molecule having a positively-charged functional group, a molecule having a hydrophobic domain, a molecule having an amine-containing ligand, a molecule having a carboxylic acid group and a molecule having an oxygen acceptor.

5. The complex according to claim 1, wherein the ratio of the negative charges in the sulphated polysaccharide to the positive charges in the compensator ranges from about 1:0.05 to about 1:3.

6. The complex according to claim 1, wherein the compensator bonds to the sulphated polysaccharide through a hydrogen bond.

7. The complex according to claim 1, wherein the compensator is selected from the group consisting of lysine or a polymerized/copolymerized molecule thereof, arginine or a polymerized/copolymerized molecule thereof, histidine or a polymerized/copolymerized molecule thereof, and glutamine or a polymerized/copolymerized molecule thereof, zein, chitosan, protamine, polyethyleneimine, amine polyethylene glycol (PEG), amine-terminated poly(ethylene oxide) (PEO) and poly(epsilon-caprolactone) (PCL), oxidized dextran, polyethylene glycol (PEG), chemically-modified PEG, polydextrose, polysorbate 20, polysorbate 80, polyvinyl acetate, Pluronic® F68, Pluronic® F123, Pluronic® F127, polyvinyl alcohol, propylene glycol alginate, and p-selectin or a modification thereof.

8. The complex according to claim 1, wherein the molar ratio of the sulphated polysaccharide to the compensator ranges from about 1:0.005 to about 1:100.

9. The complex according to claim 1, wherein the hydrophobic core is a lipid, an oil, a hydrophobic polymer, or a polypeptide.

10. The complex according to claim 9, wherein hydrophobic core is selected from the group consisting of vegetable oils, labrafac, soybean oil, castor oil, olive oil, Nigella sativa oil, garlic oil, Echium oil, cottonseed oil, peanut oil, sesame oil, anise oil, cinnamon oil, coconut oil, corn oil, PEG-60 hydrogenated castor oil, polyoxyl 35 castor oil, tristearin, phosphate lipid, egg phospholipid, stearic acid, lecithin, cholesterol, hydrogenated soy phosphatidylcholine, 1,2-distearoyl-sn-glycero-3-phosphocholine (DSPC), 1,2-Distearoyl-sn-glycero-3-phosphorylethanolamine (DSPE), DSPE-PEG, 1,2-dioleoyl-3-trimethylammoniumpropane (DOTAP), dimethyldioctadecylammonium (DDA), 1,2-dimyristoylrac-glycero-3 (DMG)-PEG, polyvinyl alcohol (PVA), poly lactic acid (PLA), poly glycolic acid (PGA), poly lactic-co-glycolic acid (PLGA), and poly-L-leucine.

11. The complex according to claim 1, which comprises fucoidan, PLGA, and lysine.

12. The complex according to claim 11, wherein the molar ratio between fucoidan to PLGA is from about 1:3 to about 1:25; and/or the molar ratio between fucoidan to lysine is from about 1:40 to about 1:160.

13. The complex according to claim 1, which comprises fucoidan, PLGA, lysine, and soybean oil.

14. The complex according to claim 13, wherein the molar ratio between fucoidan to PLGA is from about 1:3 to about 1:25; the molar ratio between fucoidan to lysine is from about 1:40 to about 1:160; and/or the molar ratio between fucoidan to soybean oil is from about 1:6 to about 1:26.

15. A pharmaceutical composition comprising a complex of claim 1 and a therapeutic agent.

16. The pharmaceutical composition according to claim 15, wherein the pharmaceutical composition is in a form of an emulsion or nanoprecipitated nanoparticle.

17. The pharmaceutical composition according to claim 15, wherein the therapeutic agent is encapsulated in the complex.

18. The pharmaceutical composition according to claim 15, wherein the loading capacity of the complex for the therapeutic agent ranges from about 1% to about 30%.

19. A method comprising use of the pharmaceutical composition of claim 15 in the manufacture of a medicament for treating a disease in a subject in need of such treatment.

20. The method of claim 19, wherein the therapeutic agent is an anticancer drug and the disease is selected from the group consisting of breast cancer, lymphoma, lung cancer, bladder cancer, ovarian cancer, and pancreatic cancer.
